(19) United States
(12) Patent Application Publication
(10) Pub. No.: US 2014/0045190 A1
(43) Pub. Date: Feb. 13, 2014

(54) METHOD AND DEVICE FOR MONITORING REAL-TIME POLYMERASE CHAIN REACTION (PCR) UTILIZING ELECTRO-ACTIVE HYDROLYSIS PROBE (E-TAG PROBE)

(71) Applicants: I Ming Hsing, Kowloon (CN); Xiaoteng Luo, Kowloon (CN); Feng Xuan, Kowloon (CN)

(72) Inventors: I Ming Hsing, Kowloon (CN); Xiaoteng Luo, Kowloon (CN); Feng Xuan, Kowloon (CN)

(73) Assignee: THE HONG KONG UNIVERSITY OF SCIENCE AND TECHNOLOGY, Kowloon, Hong Kong (CN)

(21) Appl. No.: 14/112,669

(22) PCT Filed: Apr. 18, 2012
(86) PCT No.: PCT/CN2012/000531

§ 371 Date: Oct. 18, 2013

Related U.S. Application Data
(60) Provisional application No. 61/457,546, filed on Apr. 19, 2011.

(51) Int. Cl. C12Q 1/68

(52) U.S. Cl. CPC C12Q 1/686
USPC 435/6.12; 435/287.2

(57) ABSTRACT

A method for real-time electrochemical monitoring of PCR amplicons using a hydrolysis probe that is labeled with electro-active indicators and a microchip for implementing the method. The method provided is simpler and has higher specificity compared with the prior art. The electrochemical signal measured during the PCR process can be used to determine the initial amount of the target DNA. This technique can be applied in detection and quantification of nucleic acids, especially for point-of-use applications such as on-site nucleic acid-based bio-analysis.

### TECHNICAL FIELD

The present subject matter relates to a method of quantification of nucleic acids (DNA or RNA) through real-time monitoring of the PCR amplification process and a microchip device performing the method.

### BACKGROUND

There is an urgent global need for a robust DNA-based bio-analysis technology that is compatible with portable applications, in particular, point-of-care tests (Yager, P.; Domingo, G. J.; Gerdes, J.; Annu. Rev. Biomed. Eng. 2008, 10, 107-144). Real-time Polymerase Chain Reaction (PCR) simultaneously amplifying and measuring target DNAs is considered a standard technology in DNA quantification. The traditional fluorescence-based real-time PCR is well established and is widely used in DNA-based bio-analysis. However, the requirement for bulky, expensive and complex optical equipment limits its application in portable scenarios. In order to transform the real-time PCR into a technology that is compatible with portable applications, recent research efforts have been made to replace the fluorescent measurement components of the real-time PCR system with electrochemistry-based detection, because the latter provides simple instrumentation and easy miniaturization, which are essential for development of portable bio-analysis technologies and devices.

Four main mechanisms were developed and used in fluorescence-based real-time PCR technologies (Klein, D. Trends. Mol. Med. 2002, 8, 257-260). The simplest one is based on intercalating dyes that produce enhanced fluorescence when they bind to the double-stranded PCR amplicons. Molecular beacon and hybridization probe-based real-time PCR both take advantage of the increase of fluorescence when the probe(s) hybridize with the PCR amplicon, while hydrolysis probe-based real-time PCR produces detectable fluorescence only after the cleavage of the probe. In efforts to develop electrochemistry-based real-time PCR technologies, electrochemical versions of these detection mechanisms have been studied and proposed.

Since Hsing et al. first reported electrochemical real-time PCR (ERT-PCR) (Yeung, S. W.; Lee, T. M. H.; Hsing, I. M. J. Am. Chem. Soc. 2006, 128, 13374-13375; Yeung, S. W.; Lee, T. M. H.; Hsing, I. M. Anal. Chem. 2008, 80, 363-368), a number of ERT-PCR strategies have been reported, which, interestingly, are all based on detection mechanisms analogous to the fluorescent intercalator-based real-time PCR.

For example, Gong et al., Biosensens. Bioelectron, 24 (2009), 2131-2136, reported an intercalator-based method for the real-time monitoring of PCR amplicons. In their method, the decrease of diffusion coefficient of methylene blue (MB) as it is intercalated into the double-stranded PCR products is utilized to monitor the amount of PCR amplicons produced. As the PCR proceeds, an increasing number of MB is intercalated into the PCR amplicons, resulting in a reduced electrochemical signal of MB. Based on a similar mechanism, Marchal et al., J. Am. Chem. Soc., 131 (2009), 11433-11441, developed an ERT-PCR utilizing the intrinsic electro-activity of the dNTPs, assisted by red-ox catalysts Ru(bpy)₃³⁺ (with bpy=2,2′-bipyridine) or Os(bpy)₃³⁺. With the increase of PCR cycles, an increasing number of dNTPs is consumed, leading to a reduced electrochemical signal. Both of these methods do not require immobilization of probes and demonstrate sensitivity comparable to that of a fluorescence-based system. However, they both work in a signal-off format, which is more susceptible to false positive results (Xiao, Y.; Piorek, B. D.; Plaxco, K. W.; Heeger, A. J. J. Am. Chem. Soc. 2005, 127, 17990-17991; Luo, X.; Hsing, I. M. Electroanalysis, 2010, 22, 2769-2775). Furthermore, multiplexing is impossible for these methods.

Plaxco et al. developed an electrochemical immobilized “molecular beacon” method for DNA detection, based on the conformational changes of a DNA probe immobilized on the electrode surface and labeled with electro-active indicators (e.g. ferrocene, methylene blue). Upon hybridization between the immobilized probe and the target DNA, the distance of the electro-active label on the immobilized probe is significantly changed, resulting in a dramatic increase (signal-on design) (Xiao, Y.; Piorek, B. D.; Plaxco, K. W.; Heeger, A. J. J. Am. Chem. Soc. 2005, 127, 17990-17991) or decrease (signal-off design) (Fan, C.; Plaxco, K. W.; Heeger, A. J. Proc. Natl. Acad. Sci. USA 2003, 100, 9134-9137) in the electrochemical signal.

More recently, Fang et al. reported a non-immobilizing strategy for electrochemical DNA detection based on the use of a dually labeled DNA probe (Wu, J.; Huang, C.; Cheng, G.; Zhang, F.; He, P.; Fang, Y. Electrochem. Commun. 2009, 11, 177-180). The probe has a stem-loop structure and is linked with electro-active carminic acid moieties on both ends, which are close enough to form dimers, resulting in the quenching of their electro-activity. Upon hybridization with the complementary target DNA, the carminic acids are separated, regaining the ability to produce an electrochemical signal. In an alternative strategy reported by the same group, the stem-loop DNA probe is linked with a dabcyl on one end and a gold nanoparticle on the other end (Fan, H.; Xu, Y.; Chang, Z.; Xing, R.; Wang, Q.; He, P.; Fang, Y. Biosens. Bioelectron. 2010, 26, 2655-2659). The hybridization of the probe with the target DNA separates the dabcyl from the gold nanoparticle, allowing it to bind to an α-CD modified electrode. As a result, the electrochemical signal of the gold nanoparticle can be produced. These simple strategies are elegant and sensitive, but haven't been applied in real-time PCR yet.

Electrochemical versions of DNA detection based on hydrolysis probes have also been reported. Jenkins et al. demonstrated such a method using a ferrocene-labeled DNA probe and a T7 exonuclease enzyme (Hillier, S. C.; Flower, S. E.; Frost, C. G.; Jenkins, A. T. A.; Keay, R.; Braven, H.; Clarkson, J. Electrochem. Commun. 2004, 6, 1227-1232). When the DNA probe is hybridized to the target DNA, the ferrocene label on its 5′ end is removed by the double-strand specific T7 exonuclease. As the resulting ferrocene-labeled nucleotide is much smaller and carries a much lower negative charge than the ferrocene-labeled DNA probe, it will diffuse to the electrode faster and thus produce a higher electrochemical signal of ferrocene.

For the past ten years, the development of electrochemistry-based DNA analysis technologies has been focused on point-of-care applications. Hsing et al. developed a microchip-based complete DNA bioassay platform for multiplexed pathogen detection, which is capable of handling the whole process of DNA-based bio-analysis from sample preparation and DNA amplification to sequence-specific amplicon detection (Yeung, S W.; Lee, T. M. H.; Cai, H.; Hsing, I. M. Nucl. Acids Res. 2006, 34, e118). The first electrochemistry-based real-time PCR (ERT-PCR) was reported (Yeung, S. W.; Lee, T. M. H.; Hsing, I. M. J. Am. Chem. Soc. 2006, 128, 13374-13375; Yeung, S. W.; Lee, T. M. H.; Hsing, I. M. Anal. Chem. 2008, 80, 363-368). The amount of PCR amplicons being produced is monitored electrochemically through the use of a ferrocene-labeled deoxyuridine triphosphate (Fc-dUTP), which is incorporated onto the immobilized probe during the PCR, resulting in an increasing electrochemical signal of the ferrocene. This ERT-PCR shows a better sensitivity than that of a SYBR Green fluorescence-based real-time PCR platform with high concentrations of a DNA template.

However, when detecting target DNAs at low concentrations, the performance of ERT-PCR is not satisfactory enough. Compared to the fluorescence-based real-time PCR, more cycles are required before a detectable signal can be obtained. The unsatisfactory performance of the ERT-PCR in low target DNA concentration scenarios can be attributed to (1) the low efficiency of Fc-dUTP incorporation into the PCR amplicon and onto the extended probe immobilized on the electrode, (2) the low electron transfer efficiency from the incorporated Fc-dUTP through the DNA backbone to the electrode and (3) the fact that the detecting electrode also serves as a substrate on which DNA probes are immobilized and extended during the PCR, which may cause interference to the electrochemical measurements. In 2008, an immobilization-free electrochemical method was developed for the detection of sequence specific DNA and PCR amplicons, which is based on the hybridization between the target DNA and a ferrocene-labeled PNA probe in a homogeneous solution phase, eliminating the need to immobilize DNA probes on the electrode (Luo, X.; Lee, T. M. H.; Hsing, I. M. Anal. Chem. 2008, 80, 7341-7346). This method has been demonstrated to be simple, fast and easily multiplexed (Luo, X.; Hsing, I. M. Biosens. Bioelectron. 2009, 25, 803-808).

In addition, T. H. Fang et al., Biosens. Bioelectron. 24, 2009, 2131-2136, has reported real-time PCR microfluidic devices with concurrent electrochemical detection. T. Defever et al., J. Am. Chem. Soc. 131, 2009, 11433-11441, has reported real-time electrochemical monitoring of the polymerase chain reaction by mediated red-ox catalysis. T. Defever et al., Anal. Chem. 83, 2011, 1815-1821, has reported real-time electrochemical PCR with a DNA intercalating red-ox probe. Further, B. Y. Won et al., Analyst, 2011, 136, 1573-1579, has reported an investigation of the signaling mechanism and verification of the performance of an electrochemical real-time PCR system based on the interaction of methylene blue with DNA.

Regarding patent/application publications, J. Lee et al., U.S. Pat. No. 7,135,294 B2, has developed a method for real-time detection of PCR products using an electrical signal. During PCR, nucleotides are incorporated into the amplicon, resulting in decrease of the electrical mobility of the PCR mixture. Therefore, as the PCR proceeds the impedance of the PCR mixture increases. Thus, the PCR amplification process can be monitored in real-time by measuring the impedance of the solution. A. Heller et al., U.S. Patent Application Publication No. 2002/0001799 A1, has described rapid amperometric verification of PCR amplification of DNA in a small sample of the PCR product. Jung-im Han, U.S. Patent Application Publication No. 2005/0191686 A1, describes a micro PCR device, a method for amplifying nucleic acids using the micro PCR device and a method for measuring concentration of PCR products using the micro PCR device. Further, I. M. Hsing et al., US Patent Application Publication No. 2010/0184028 A1, has described a method and a system for real time quantification and monitoring of nucleic acids using electroconductive or electrochemically active labels.

However, there is still a need in the art for a real-time ERT-PCR method for quantifying nucleic acid that is simpler, immobilization-free and has a higher specificity.

### SUMMARY

The present subject matter describes a method for real-time electrochemical measuring of the amounts of the amplicon in PCR, using a DNA probe labeled with one or more electro-active indicators (called the eTaq probe) and an electrode with a negatively charged surface. The eTaq probe is complementary to part of the PCR amplicon and is hydrolyzed during the extension of the PCR primers by a DNA polymerase with exonuclease activity. The resultant electro-active nucleotides have a higher diffusion coefficient and less negative charge, leading to an enhanced electrochemical signal. The increase of the electrochemical signal over PCR cycles can be used to determine the initial amount of the target DNA template.

The present method is simpler, requiring no probe immobilization, and has a higher specificity, compared to the prior art. Thus, the present method can be applied in detection and quantification of nucleic acids, especially for point-of-use applications, such as on-site nucleic acid-based bio-analysis. Particularly as compared with the first developed ERT-PCR by Hsing et al., the present eTaq-based ERT-PCR method has no problems of low Fc-dUTP incorporation and electron-transfer efficiency, while it takes advantages of the hydrolysis of the eTaq probe and the diffusion-controlled electrochemical reaction of the released ferrocene-labeled dUTP. In the present eTaq-based ERT-PCR, the hydrolysis of the eTaq probe occurs in the solution phase or on a second substrate rather than on the detection electrode, avoiding interference to the electrochemical measurements.

Accordingly, one aspect of the present subject matter is directed to a method of electrochemically monitoring and/or quantifying the amplified nucleic acid products by polymerase chain reaction (PCR) (or PCR amplicon) in real-time or after each PCR thermal cycle, comprising: contacting a sample comprising a target nucleic acid with a single-stranded hydrolysis DNA probe labeled with at least one electroactive indicator, adding a PCR enzyme, such as a DNA polymerase with 5′-3′ exonuclease activity, under conditions effective for PCR amplification to occur, adding an electric potential, and detecting or measuring in real-time or after each PCR thermal cycle an electric signal produced by the electroactive indicator and/or quantifying the amount of nucleic acid present in the sample.

The single-stranded hydrolysis DNA probe is complementary to a region within the PCR amplicon and has a 3′ end that can not be extended. In an embodiment, the hydrolysis DNA probe is phosphorylated at its 3′ end. In another embodiment, the hydrolysis DNA probe has at least one base at its 3′ end that is not complementary to the PCR amplicon. The probe can be used multiplexing. Either one or multiple electroactive indicators can be labeled onto the probe. Preferably, the electroactive indicator(s) is ferrocene or methylene blue. The electric signal can be detected or measured with a conductive electrode(s) with a negatively charged surface comprising, e.g., indium tin oxide, gold, platinum, carbon and/or magnetic particles. In an embodiment, the electrodes can be interdigitated array (IDA) electrodes. The electroactive probe can be hydrolyzed by a DNA polymerase and the amount hydrolyzed increases during the PCR thermal cycling process, in proportion to the amount of amplicons produced in the PCR thermal cycling process.

Another aspect of the present subject matter is directed to a microchip for implementing the presently provided method, comprising an electrochemically conductive electrode(s) and a support adapted to receive a solution comprising nucleic acid. The PCR reaction can be performed in a micro-chamber of the microchip, preferably made of Si. The microchip is preferably produced between anodically bonded Si and glass substrates. The microchip can contain a metal-based temperature sensor(s) and a micro heater(s) integrated thereon, preferably to control the temperature during the PCR reaction. A detection electrode(s) can be patterned and integrated on the microchip and a surface of the electrode(s) can preferably comprise indium tin oxide, gold, platinum, carbon and/or magnetic particles. The electrode(s) can be used to detect or measure the electrochemical signal produced by the method in proportion to the amount of PCR amplicons produced.

### BRIEF DESCRIPTION OF THE DRAWINGS

Various embodiments will now be described in detail with reference to the accompanying drawings.

FIG. 1 illustrates a scheme for an embodiment of the present subject matter. FIG. 2 illustrates one embodiment of the present subject matter using human genomic DNA (male) as the template and amplifying a 137-bp segment of the human sex-determining region Y (SRY). The graphs of FIG. 2(a) are differential pulse voltammetry (DPV) scans in the real-time PCR utilizing electroactive hydrolysis probe after 0, 5, 10, 20, 30 or 40 cycles and the graph of FIG. 2(b) is a plot of peak current intensity in the DPV scans against PCR cycle numbers.

FIG. 3 is a schematic illustration of electrochemical real-time PCR using a hydrolysis probe labeled with multiple electroactive indicators.

FIG. 4 is a schematic illustration of the signal amplification mechanism of interdigitated array electrodes.

FIG. 5 is schematic illustration of multiplex electrochemical real-time PCR using multiple electroactive hydrolysis probes.

### DETAILED DESCRIPTION

Throughout the application, various embodiments are described using the term “comprising”; however, it will be understood by one of skill in the art, that in some specific instances, an embodiment can alternatively be described using the language “consisting essentially of” or “consisting of.”

For purposes of better understanding the present subject matter and in no way limiting the scope of the present subject matter, unless otherwise indicated, all numbers expressing quantities, percentages or proportions, and other numerical values used herein, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

The term “a” or “an” as used herein includes the singular and the plural, unless specifically stated otherwise. Therefore, the term “a,” “an” or “at least one” can be used interchangeably in this application.

Other terms as used herein are meant to be defined by their well-known meanings in the art.

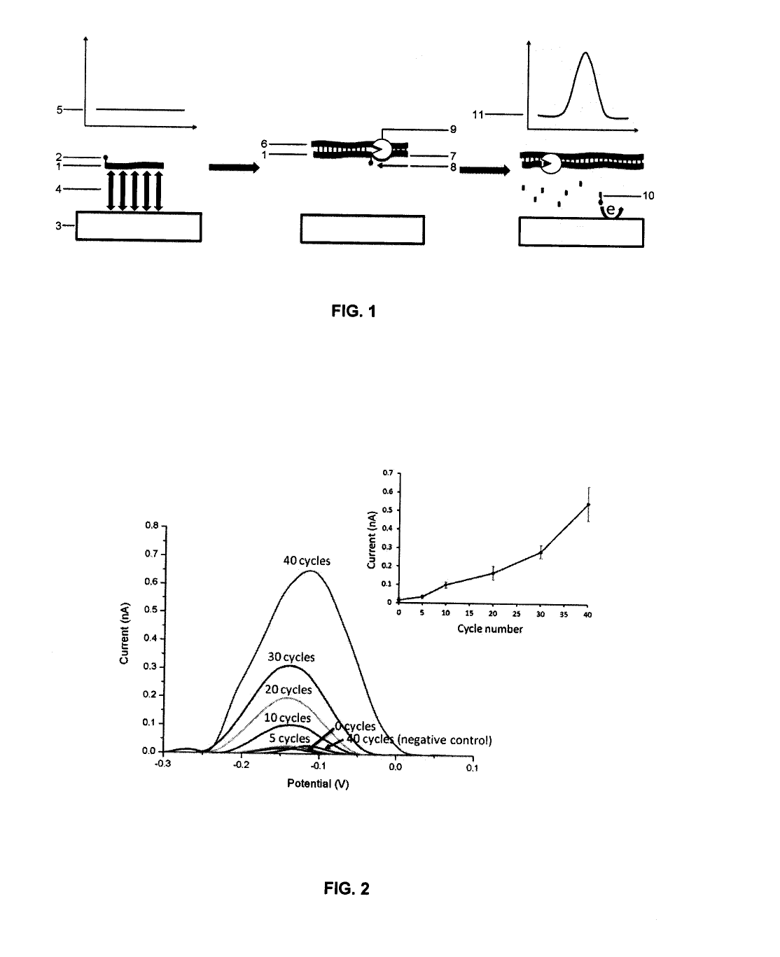

Referring now to the embodiments of the present subject matter in more detail, FIG. 1 shows a schematic illustration of an embodiment of the present subject matter where a DNA oligonucleotide 1 (also called the eTaq probe) labeled with an electroactive indicator 2 (e.g. ferrocene, methylene blue) and an electrode 3 (e.g. indium tin oxide electrode) with a negatively-charged surface are used. Before PCR, due to the electrostatic repulsion 4 between the negative DNA backbone and the negative electrode surface, the eTaq probe 1 is prevented from approaching the electrode, resulting in a negligible electrochemical signal 5 of the electroactive indicator 2. When the PCR amplicon 6 is produced, both the eTaq probe 1 and the PCR primer 7 anneal to the complementary regions within the PCR amplicon 6. As the PCR primer 7 is elongated in the extension 8 catalyzed by the DNA polymerase 9 with exonuclease activity, the eTaq probe 1 is hydrolyzed by the DNA polymerase 9, releasing a nucleotide 10 labeled with the electroactive indicator 2. As the negative charge on the electroactive nucleotide 10 is much less than that on the eTaq probe 1, it is possible for the electroactive nucleotide 10 to diffuse to the electrode surface, producing a detectable electrochemical signal 11.

In more detail, still referring to the schematic illustration of FIG. 1 for an embodiment of the present subject matter, the electroactive indicator 2 associated with the eTaq probe 1 can not diffuse to the electrode 3 because of the electrostatic repulsion between the negatively charged DNA backbone and the negatively charged electrode surface, resulting in a negligible electrochemical signal of the electroactive indicator 2, as reported by Luo, et al., Anal. Chem., 80, 7341-7346 (2008) and Luo, et al., Electroanalysis, 22, 2769-2775 (2010). During the annealing step of the PCR cycles, both the eTaq probe 1 and the PCR primer 7 anneal to the complementary regions within the PCR amplicon, with the PCR primer 7 at the upper stream. The PCR primer 7 is then extended along the PCR amplicon 6 by the DNA polymerase 9. When the DNA polymerase 9 encounters the eTaq probe 1, the eTaq probe 1 is hydrolyzed because of the 5′-3′ exonuclease of the DNA polymerase 9. That is, the PCR enzyme is a DNA polymerase with 5′-3′ exonuclease activity.

The mechanism of hydrolysis of the probe as a result of the extension of the PCR primer is the same as utilized in the TaqMan® probe-based fluorescent real-time PCR developed by Mayrand (U.S. Pat. No. 6,395,518 B1). In Mayrand's method, the hydrolysis probe is labeled with a fluorescent molecule at one end and a quencher molecule at the other end. The hydrolysis of the probe separates the fluorescent molecule from the quencher molecule, resulting in the emission of the fluorescent signal. In the present subject matter, the hydrolysis cleaves the eTaq probe 1 into an electroactive nucleotide 10. The negative charge on the electroactive nucleotide 10 is much less than that on the eTaq probe 1, therefore it is possible for the electroactive nucleotide 10 to diffuse to the electrode 3, producing a detectable electrochemical signal 11. It is observed that a higher electrochemical signal is detected when an electro-active DNA probe is hydrolyzed, as reported by Jenkins et al., Bioelectrochem., 63 (2004), 307-310 and Jenkins et al., Electrochem. Commun., 6 (2004), 1227-1232, where the hydrolysis of DNA probes by nucleases were utilized for the detection of DNA and nuclease, but not for real-time PCR.

In further details, still referring to the schematic illustration of FIG. 1 for an embodiment of the present subject matter, the 3′ end of the eTaq probe 1 is phosphorylated, in order to prevent the elongation of the eTaq probe 1 during the PCR, as the elongation of the eTaq probe 1 may cause interference to the PCR and reduce amplification efficiency.

Referring now to a demonstration of an embodiment of the present subject matter in FIG. 2, a 137-bp segment of the human sex-determining region Y (SRY) is amplified from human genomic DNA (male). A methylene blue-labeled DNA is used for the eTaq probe (MB-eTaq). The MB-eTaq probe is phosphorylated at its 3′-end to prevent its elongation during the PCR. During the annealing step of the PCR thermal cycling, both the MB-eTaq probe and the PCR forward primer hybridized to the complementary regions of the denatured PCR amplicon. With the elongation of the primer, the MB-eTaq probe is hydrolyzed into an electro-active nucleotide (MB-dATP), resulting in an enhanced electrochemical signal. As the PCR proceeds, more of the PCR amplicon, and therefore more of the electro-active nucleotide, is produced, and the electrochemical signal measured increases correspondingly, thus real-time monitoring of the amplification of the PCR amplicon is made possible. As shown in FIG. 2, ascending signals of the methylene blue are measured at increasing cycle numbers. In the negative control without template DNA, negligible signal is measured even after 40 cycles, proving the high specificity of the present method.

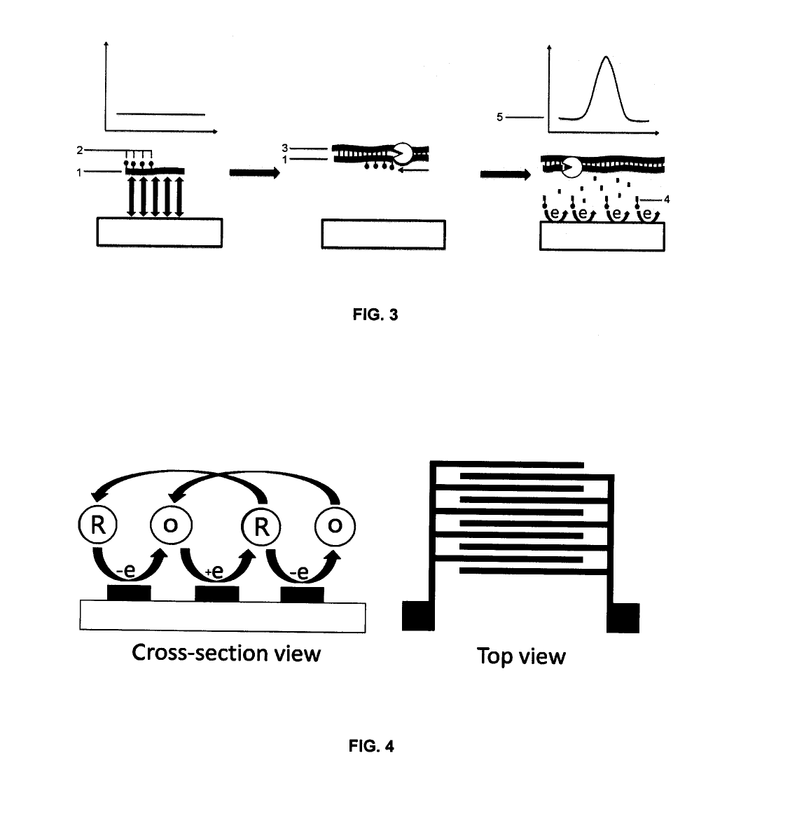

Referring to FIG. 3 illustrating a scheme for electrochemical real-time PCR using a hydrolysis probe labeled with multiple electroactive indicators, a hydrolysis probe 1 can be labeled with multiple electroactive indicators 2, such as methylene blue and ferrocene. Therefore, as each PCR amplicon 3 is produced, multiple electroactive nucleotides 4 are released, resulting in an amplified electrochemical signal 5 and improved detection sensitivity.

Referring to FIG. 4 illustrating a scheme for the signal amplification mechanism of interdigitated array (IDA) electrodes, IDA electrodes are a recently developed kind of electrodes that can produce an amplified electrochemical signal based on subjecting the electro-active species to multiple red-ox cycles. Because of the narrow gap between the interdigitated electrodes, an electro-active species that is oxidized (or reduced) at one electrode can be reduced (or oxidized) at an adjacent electrode when different potentials are applied on the interdigitated electrodes, forming a red-ox cycle. The same molecule undergoes multiple red-ox cycles before it diffuses away from the electrodes, resulting in a strong amplification of the electrochemical signal. IDA electrodes are only applicable to electrochemical red-ox reactions that are diffusion-controlled, that is, the electro-active species should be able to diffuse freely between the oxidizing electrodes and the reducing electrodes. As the present eTaq-based ERT-PCR method measures the electrochemical signal produced by Fc-dUTP, which diffuses from the solution to the electrode surface, it is a perfect match with IDA electrodes. Therefore, IDA electrodes can be applied to the present eTaq-based ERT-PCR method to obtain an improved detection sensitivity.

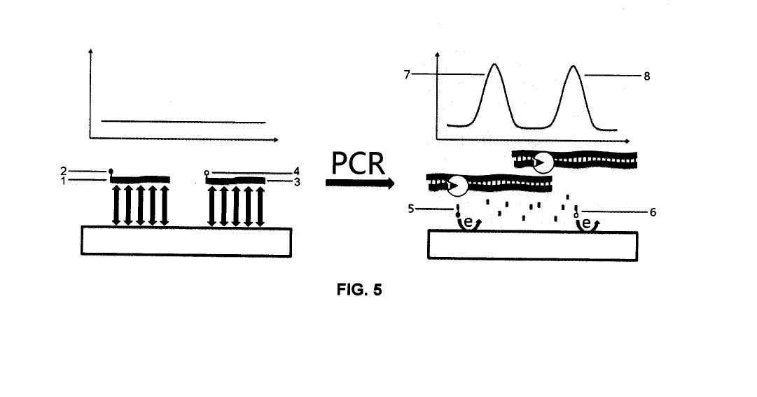

Referring to FIG. 5, multiplexed electrochemical real-time PCR can be realized by using multiple hydrolysis probes labeled with electro-active indicators of different red-ox potentials. Ferrocene (Fc) and methylene blue (MB) are two electro-active indicators that have distinct red-ox peaks. As demonstrated in FIG. 5, in multiplexed eTaq-based ERT-PCR, a hydrolysis probe 1 labeled with Fc 2 and a hydrolysis probe 3 labeled with MB 4, with sequences complementary to respective PCR amplicons, are added to the same PCR mixture. As the PCR amplicons are produced, the corresponding hydrolysis probes are hydrolyzed, releasing Fc-labeled dNTP 5 and MB-labeled dNTP 6, resulting in the electrochemical signal of Fc 7 and the signal of MB 8. Thus, the signal intensity of Fc and MB reflects the initial amounts of the corresponding target DNA templates, realizing multiplexed electrochemical real-time PCR using one detection electrode.

Also provided herein is a microchip for implementing the presently provided method, comprising an electrochemically conductive electrode(s) and a support adapted to receive a solution comprising nucleic acid. This microchip used for implementing the present eTaq-based ERT-PCR is similar to but different from the chip electrodes reported by Hsing et al., Anal. Chem. 80, 2008, 7341, the content of which is incorporated herein by reference in its entirety.

In particular, the PCR reaction can be performed in a micro-chamber of the microchip, preferably made of Si. The micro-chamber can be preferably produced between anodically bonded Si and glass substrates. The microchip can contain a metal-based temperature sensor(s) and a micro heater(s) integrated thereon, preferably to control the temperature during the PCR reaction. A detection electrode(s) can be patterned and integrated on the microchip and a surface of the electrode(s) can preferably comprise indium tin oxide, gold, platinum, carbon and/or magnetic particles. The electrode(s) can be used to detect or measure the electrochemical signal produced by the method in proportion to the amount of PCR amplicons produced. In this regard, the current of the electrochemical signal can be correlated to the amount of amplified nucleic acid products.

Kits similar to the ones described for the prior methods can also be contemplated to implement the present method, and may contain all necessary components for the practice of the present subject matter, such as primers, microchip, electrodes, PCR reagents, and the like. When provided immediately prior to its utilization the kits may also contain a labeled marker(s), and other custom made reagents.

The advantages of the present subject matter include, without limitation, improving the specificity of real-time PCR, requiring no probe immobilization and allowing easy multiplexing. The presently provided method is easy to perform without sophisticated instruments and complicated processes and requires generally no more than a few hours to complete. In one broad embodiment, the present subject matter is a method to determine the presence and amount of a target nucleic acid (DNA or RNA). As the electrochemical method has the advantages of easy miniaturization, effortless operation, simple instrumentation and low cost, the present subject matter is especially suitable for portable nucleic acid-based bio-analysis.

### EXAMPLES

The following preparations and examples are given to enable those skilled in the art to more clearly understand and to practice the present subject matter. They should not be considered as limiting the scope of the claims, but merely as being illustrative and representative thereof.

All the reagents used herein were of analytical grade unless described otherwise, and deionized water was used throughout the experiment. The microchip used for electrochemical measurement was similar to the chip electrodes reported by Hsing et al., Anal. Chem. 80, 2008, 7341, the content of which is incorporated herein by reference in its entirety, and its fabrication was done in Nanoelectronics Fabrication Facility (NFF) of The Hong Kong University of Science and Technology. Electrochemical measurements were performed with an Autolab PGSTAT30 potentiostat/galvanostat (Eco Chemie). PCR was performed with a C1000TM thermal cycler (Bio-Rad).

### Example 1

### Electrochemical Real-Time Monitoring of PCR Amplification of a 137-bp Target DNA Using an Electro-Active Hydrolysis Probe

A 137-bp segment of the human sex-determining region Y (SRY) was amplified from human genomic DNA (male) (Promega). The sequences of the PCR primers were 5′-TGG CGA TTA AGT CAA ATT CGC-3′ (SEQ ID NO: 1) (forward) and 5′-CCC CCT AGT ACC CTG ACA ATG TAT T-3′ (SEQ ID NO: 2) (reverse) (Invitrogen). A 26-mer methylene blue-labeled DNA with a sequence of MB-5′-AGC AGT AGA GCA GTC AGG GAG GCA GA-3′-phos (SEQ ID NO: 3) (BioSearch) was used as the eTaq probe (MB-eTaq). The MB-eTaq probe was phosphorylated at its 3′-end to prevent its elongation during the PCR. PCR mixtures with and without human genomic DNA (male) were prepared. The positive PCR mixture containing 1×AmpliTaq Gold 360, 2 mM MgCl₂, 0.2 mM dNTPs, 1 μM forward primer, 1 μM reverse primer, 1.6×10⁶ copies/μL Human genomic DNA (male), 1 μM MB-eTaq and 0.1 U/μL AmpliTaq Gold 360 DNA polymerase was prepared in AmpliTaq Gold 360 buffer (Applied Biosystems). PCR mixture without the human genomic DNA (male) was prepared as the negative control. This PCR solution was subjected to the following thermal cycling process: initial denaturation at 94° C. for 10 min; 0, 5, 10, 20, 30 or 40 cycles at 94 ° C. for 10 sec and 60° C. for 60 sec; final extension at 60° C. for 5 min. After certain cycle numbers, 2 μL of the PCR mixture was pipetted onto a chip containing an ITO working electrode, a Pt counter electrode and a Pt pseudo-reference electrode, and DPV measurement was performed immediately. As the cycle number increased, higher peaks specific to MB were observed in the DPV scans. The DPV scan results are shown in FIG. 2(a) and a plot of peak current intensity in the DPV scans against cycle numbers is shown in FIG. 2(b).

### Example 2

### Electrochemical Real-Time Monitoring of PCR Amplification Using a Hydrolysis Probe Labeled with Multiple Electroactive Indicators

Using the same materials and methods of Example 1 except that the hydrolysis probe is labeled with multiple electroactive indicators, electrochemical real-time monitoring of PCR amplification is performed as shown in FIG. 3. The hydrolysis probe 1 in FIG. 3 is labeled with multiple electroactive indicators 2 (multiple-MB-eTaq probe). As each PCR amplicon 3 is produced, multiple electroactive nucleotides 4 are released, resulting in an amplified electrochemical signal 5 and improved detection sensitivity.

### Example 3

Electrochemical real-time monitoring of PCR amplification using an interdigitated array (IDA) electrode.

The present eTaq-based ERT-PCR amplification is performed using interdigitated array (IDA) electrodes, as illustrated in FIG. 4. Because of the narrow gap between the interdigitated electrodes, an electro-active species that is oxidized (or reduced) at one electrode can be reduced (or oxidized) at an adjacent electrode when different potentials are applied on the interdigitated electrodes, forming a red-ox cycle. The electro-active species undergoes multiple red-ox cycles before it diffuses away from the electrodes, resulting in a strong amplification of the electrochemical signal. IDA electrodes are only applicable to electrochemical red-ox reactions that are diffusion-controlled, that is, the electro-active species should be able to diffuse freely between the oxidizing electrodes and the reducing electrodes. Since the present eTaq-based ERT-PCR method measures the electrochemical signal produced by Fc-dUTP which diffuses from the solution to the electrode surface, using the IDA electrodes can obtain an improved detection sensitivity.

### Example 4

### Multiplexed Electrochemical Real-Time PCR with Multiple Electroactive Hydrolysis Probes

The present eTaq-based ERT-PCR is performed using multiple hydrolysis probes labeled with electro-active indicators of different red-ox potentials, such as, ferrocene (Fc) and methylene blue (MB). As demonstrated in FIG. 5, to perform the multiplexed eTaq-based ERT-PCR, a hydrolysis probe 1 is labeled with Fc 2 and the other hydrolysis probe 3 is labeled with MB 4, both of which probes have sequences complementary to respective PCR amplicons, and they are added to the same PCR mixture. As the PCR amplicons are produced, the corresponding hydrolysis probes are hydrolyzed, releasing Fc-labeled dNTP 5 and MB-labeled dNTP 6, resulting in the electrochemical signal of Fc 7 and the signal of MB 8. The signal intensity of Fc and MB reflects the initial amounts of the corresponding target DNA templates, realizing multiplexed electrochemical real-time PCR using one detection electrode.

While the foregoing written description of the present subject matter enables one of ordinary skill to make and use what is considered presently to be the best mode thereof, those of ordinary skill will understand and appreciate the existence of variations, combinations, and equivalents of the specific embodiment, method, and examples herein. The present subject matter should therefore not be limited by the above described embodiment, method, and examples, but by all embodiments and methods within the scope and spirit of the present subject matter.

We claim:

1. A method of electrochemically monitoring and/or quantifying the amplified nucleic acid products by polymerase chain reaction (PCR) or PCR amplicon in real-time or after each PCR thermal cycle, comprising: contacting a sample comprising a target nucleic acid with a single-stranded hydrolysis DNA probe labeled with at least one electroactive indicator, adding a PCR enzyme under conditions effective for PCR amplification to occur, adding an electric potential, and detecting or measuring in real-time or after each PCR thermal cycle an electric signal produced by the electroactive indicator and/or quantifying the amount of nucleic acid present in the sample.

2. The method of claim 1, wherein the single-stranded hydrolysis DNA probe has a 3′ end that can not be extended.

3. The method of claim 2, wherein the single-stranded hydrolysis DNA probe is phosphorylated at its 3′ end.

4. The method of claim 2, wherein the single-stranded hydrolysis DNA probe has at least one base at its 3′ end that is not complementary to the PCR amplicon.

5. The method of claim 1, wherein the single-stranded hydrolysis DNA probe is complementary to a region within the PCR amplicon.

6. The method of claim 1, wherein the PCR enzyme is a DNA polymerase with 5′-3′ exonuclease activity.

7. The method of claim 1, wherein the electric signal is detected or measured with a conductive electrode(s) with a negatively charged surface.

8. The method of claim 1, wherein the surface of the electrode(s) comprises indium tin oxide, gold, platinum, carbon or magnetic particles.

9. The method of claim 7, wherein the electrodes are interdigitated array (IDA) electrodes.

10. The method of claim 1, wherein the single-stranded hydrolysis DNA probe labeled with an electroactive indicator(s) is hydrolyzed by a DNA polymerase and the amount hydrolyzed increases during the PCR thermal cycling process.

11. The method of claim 10, wherein electroactive nucleotides are accumulated in proportion to the amount of amplicons produced in the PCR thermal cycling process.

12. The method of claim 1, wherein the current of the electric signal is correlated to the amount of amplified nucleic acid products.

13. The method of claim 1, wherein the electroactive indicator is ferrocene or methylene blue.

14. The method of claim 1, wherein multiple electroactive indicators are labeled onto the probe.

15. The method of claim 1, wherein multiple hydrolysis DNA probes are used which are labeled with different electroactive indicators.

16. A microchip for implementing the method of claim 1, comprising an electrochemically conductive electrode(s) and a support adapted to receive a solution comprising nucleic acid.

17. The microchip of claim 16, wherein the PCR reaction is performed in a micro-chamber.

18. The microchip of claim 17, wherein the micro-chamber is produced between anodically bonded Si and glass substrates.

19. The microchip of claim 16, wherein a metal-based temperature sensor(s) and a micro heater(s) are integrated on the microchip.

20. The microchip of claim 19, wherein the integrated heaters and sensors are used to control the temperature during the PCR reaction.

21. The microchip of claim 16, wherein a detection electrode(s) are patterned and integrated on the microchip.

22. The microchip of claim 21, wherein a surface of the electrode(s) comprises indium tin oxide, gold, platinum, carbon or magnetic particles.

23. The microchip of claim 22, wherein the electrode(s) is used to detect or measure the electrochemical signal which reflects the amount of PCR amplicons produced.
